(19) United States
(12) Patent Application Publication
(10) Pub. No.: US 2019/0008743 A1
(43) Pub. Date: Jan. 10, 2019

(54) POLYMER COMPOSITE PARTICLES CONTAINING MUCH OF ORGANIC SUNSCREEN AGENTS AND THE METHOD FOR PREPARING THEREOF

(71) Applicant: SUNJIN BEAUTY SCIENCE CO., LTD, Ansan-si (KR)

(72) Inventors: Sung-Ho LEE, Seoul (KR); Jang-Ho PARK, Suwon-si (KR); Yang-Bae YOON, Ansan-si (KR); Deuk-Yong SEONG, Siheung-si (KR)

(21) Appl. No.: 15/557,207

(22) PCT Filed: Jun. 24, 2016
(86) PCT No.: PCT/KR2016/006735

§ 371 Date: Sep. 11, 2017

(30) Foreign Application Priority Data
Mar. 30, 2016 (KR) 10-2016-0038283

(51) Int. Cl. A61K 8/49; A61K 8/81; A61K 8/02; A61K 8/35; A61K 8/19; A61Q 17/04

(52) U.S. Cl. CPC A61K 8/4966; A61K 8/8147; A61K 8/0283; A61K 2800/412; A61K 8/19; A61Q 17/04; A61K 2800/95; A61K 8/35

(57) ABSTRACT

The present invention relates to polymer composite particles containing much organic sunscreen agents and a method of preparing the polymer composite particles, and more particularly to polymer composite particles containing much organic sunscreen agents that are spherical polymer composite particles in which solid organic Sunscreen agents are embedded between polymer matrixes by 40 to 50 wt %, include a significantly larger amount of the organic sunscreen agents than polymer composite particles of related art, have excellent light stability despite presence of a large amount of organic sunscreen agents, are superior not only in UV-A region but also UV-B region, are excellent in skin stability and feeling of use when applied to cosmetics, and can significantly improve sunscreen function, and a method of preparing the polymer composite particles.

### BACKGROUND

### 1. Field

The present invention relates to polymer composite particles containing much organic sunscreen agents and a method of preparing polymer composite particles, and more particularly to polymer composite particles containing much organic sunscreen agents that are spherical polymer composite particles in which solid organic sunscreen agents are embedded between polymer matrixes by 40 to 50 wt %, include a significantly larger amount of the organic sunscreen agents than polymer composite particles of related art, have excellent light stability despite presence of a large amount of organic sunscreen agents, are superior not only in UV-A region but also UV-B region, are excellent in feeling of use when applied to cosmetics, and can significantly improve sunscreen function, and a method of preparing the polymer composite particles.

### 2. Description of the Related Art

Recently, as the irradiation amount of ultraviolet rays of an earth surface increases due to destruction of an ozone layer, a market of products which is mainly used for ultraviolet ray blocking is growing year by year, and cosmetics using composite particles containing organic sunscreen agents as one of the above products are used.

Most organic sunscreen agents have poor stability against heat and light, and particularly, avobenzone has a problem that the avobenzone has a limited use method due to poor solubility in solvents and causes skin troubles. In order to overcome the problem, the present applicant filed Korean Patent Application No. 10-2007-0083469 entitled polymer composite particles for cosmetic composition containing an organic sunscreen agent and a method of preparing the same and it is patented. However, there is room for further improvement in meeting sunscreen function and light stability requirements. Therefore, development of composite particles capable of simultaneously enhancing sunscreen function, light stability, and skin stability and feeling when applied to cosmetics is requested.

### SUMMARY OF THE DISCLOSURE

An object of the present disclosure is to provide polymer composite particles which include a significantly larger amount of the organic sunscreen agents than polymer composite particles of related art, have excellent light stability despite presence of a large amount of organic sunscreen agents, are superior not only in UV-A region but also UV-B region, are excellent in feeling of use when applied to cosmetics, and can significantly improve sunscreen function, and a method of preparing the polymer composite particles.

In addition, another object of the present invention is to provide a cosmetic composition including polymer composite particles containing organic sunscreen agents as an active ingredient.

In order to solve the above problem, the present invention provides a polymer composite particle including a spherical particle having a solid organic sunscreen agent which is embedded between polymer matrixes by 40 to 50 wt %.

It is preferable that a size of the composite particle is 1 to 100 μm.

It is preferable that the organic sunscreen agent includes at least two kinds of sunscreen agents.

It is preferable that the organic sunscreen agent includes 50 to 90 parts by weight of avobenzone and 10 to 50 parts by weight of a triazine-based sunscreen agent.

It is preferable that the triazine-based sunscreen agent is selected from groups which are formed of BEMT (Bis-Ethylhexyloxyphenol Methoxyphenyl Triazine), Ethylhexyl Triazone, and diethylhexyl butamido triazone.

In addition, the present invention provides a method of preparing polymer composite particles, the method including a) dissolving and dispersing an organic sunscreen agent in a radical polymerizable monomer and a multifunctional crosslinking monomer in an emulsifying agent and adding an initiator to prepare an oil phase, b) mixing and emulsifying an aqueous phase containing an ionic imparting substance, a dispersion stabilizer, and water in the emulsifying agent to form droplets, c) polymerizing the droplets to prepare polymer composite particles, and d) washing and drying the polymer composite particles which are prepared.

It is preferable that the ionic-imparting substance is 10 to 30 parts by weight per 100 parts by weight of a sum of the radical polymerizable monomer and the multifunctional crosslinking monomer.

It is preferable that one or more of the ionic imparting substance are selected from a group which is formed of NaNO₃, NaCl, NaOH, Na₂SO₄, NaNO₂, Na₂CO₃, Na₃PO₄, KNO₃, KCl, KOH, K₂SO₄, K₂CO₃, K₃PO₄, NH₄NO₃, NH₄Cl, NH₄OH, (NH₄)₂SO₄, (NH₄)₃PO₄, Mg(NO₃)₂, MgCl₂, Ca(NO₃)₂, CaCl₂, Ba(NO₃)₂, and BaCl₂.

In addition, the present invention provides a cosmetic composition including the polymer composite particles as an active ingredient.

Polymer composite particles containing much organic sunscreen agents according to the present invention include a significantly larger amount of the organic sunscreen agents than polymer composite particles of related art since 40 to 50% by weight of a solid organic sunscreen agent is embedded in the matrix of spherical polymer particles, have excellent light stability despite presence of a large amount of organic sunscreen agents, are superior not only in UV-A region but also UV-B region, are excellent in skin stability and feeling of use when applied to cosmetics, and can significantly improve sunscreen function.

### BRIEF DESCRIPTION OF THE DRAWINGS

FIG. 1 is a photograph of a polymer composite particle prepared according to an embodiment of the present invention.

FIG. 2 is a photograph of the polymer composite particle prepared according to a comparative example.

FIG. 3 is a photograph of a result of an iron powder test of the polymer composite particle prepared according to the embodiment of the present invention.

FIG. 4 is a photograph of an iron powder test result of the polymer composite particle prepared according to the comparative example of the present invention.

### DETAILED DESCRIPTION OF THE EMBODIMENT

Hereinafter, the present invention will be described in detail.

The polymer composite particles according to the present invention are spherical polymer composite particles in which a solid organic sunscreen agent is embedded between polymer matrixes by 40 to 50 wt %. The spherical polymer particles according to the present invention have a structure in which 40 to 50% by weight of a solid organic sunscreen agent is embedded in an inner matrix of the spherical polymer particles, have an excellent spherical ratio exceeding 90% spherical ratio, include a significantly larger amount of the organic sunscreen agents than polymer composite particles of related art of including organic sunscreen agents in an amount not more than 30% by weight based on a total weight of the polymer composite particles.

The polymer composite particles can be prepared in various sizes and have an average particle diameter of 1 to 100 μm, preferably 2 to 20 μm. Within the above range, light stability of the composite particles and optimal feeling when applied to a cosmetic composition can be obtained.

Preferably, the organic sunscreen agent may include at least two sunscreen agents. In the present invention, not only a UV-A region but also a UV-B region having excellent sunscreen function can be provided at the same time by including at least two kinds of sunscreen agents in a large amount. The organic sunscreen agent may be a known sunscreen agent present in a solid form at room temperature. More preferably, the organic sunscreen agent includes avobenzone. Avobenzone has an excellent UV-blocking property against the UV-A region. The organic sunscreen agent preferably contains 50 to 90 parts by weight of avobenzone and 10 to 50 parts by weight of a triazine-based sunscreen agent. The triazine-based sunscreen agent may be at least one selected from a group formed of BEMT (Bis-Ethylhexyloxyphenol Methoxyphenyl Triazine), Ethylhexyl Triazone, and diethylhexyl butamido triazone. By including the triazine-based sunscreen agent within the above range, the polymer composite particle according to the present invention not only improves light stability of avobenzone, but also enhances sunscreen properties against an ultraviolet UV-B region.

The polymer composite particles can be prepared by including a) dissolving and dispersing an organic sunscreen agent in a radical polymerizable monomer and a multifunctional crosslinking monomer in an emulsifying agent and adding an initiator to prepare an oil phase, b) mixing and emulsifying an aqueous phase containing an ionic imparting substance, a dispersion stabilizer, and water in the emulsifying agent to form droplets, c) polymerizing the droplets to prepare polymer composite particles, and d) washing and drying the polymer composite particles which are prepared.

### a) Preparing Oil Phase in Emulsifier

The organic sunscreen agent in a) according to the present invention may block ultraviolet rays through absorption and use of an organic sunscreen agent existing in a solid state at room temperature. Preferably, at least two kinds of organic sunscreen agents are used in combination with the organic sunscreen agent. Preferably, the organic sunscreen agent contains avobenzone, and more preferably, the organic sunscreen agent contains 50 to 90 parts by weight of avobenzone and 10 to 50 parts by weight of a triazine-based sunscreen agent.

The organic sunscreen agent is preferably contained in an amount of 80 to 100 parts by weight based on 100 parts by weight of a radical polymerizable monomer and a multifunctional crosslinking monomer.

The radical polymerizable monomer of the present invention is copolymerizable with a vinyl-based crosslinking monomer and is not limited in particular. Specific examples of the radical polymerizable monomer include aromatic vinyl monomers such as styrene, methylstyrene, ethylstyrene, fluorostyrene, chlorostyrene, vinyltoluene, acrylic vinyl monomers such as methyl (meth) acrylate, ethyl (meth) acrylate, butyl (meth) acrylate, octyl (meth) acrylate, stearyl (meth) acrylate, benzyl (meth) acrylate, gylcidyl (meth) acrylate, fluoroethyl acrylate and the like, vinyl cyanide monomers such as (meth) acrylonitrile and the like. The monomers may be used singly or in combination of two or more.

The multifunctional crosslinking monomer is used for crosslinking particles, and a multifunctional vinyl-based crosslinking monomer capable of copolymerizing with the radical polymerizable monomers is used. Specific examples thereof include divinylbenzene, ethylene glycol dimethacrylate, diethylene glycol methacrylate, triethylene glycol dimethacrylate, trimethylene propane trimethacrylate, 1,3-butanediol methacrylate, 1,6-hexanediol dimethacrylate, allyl (meth) acrylate, tri (meth) acrylate, and the like. These may be used singly or in combination of two or more.

Preferably, a content ratio of the radical polymerizable monomer to the multifunctional crosslinking monomer is 5 to 95 parts by weight: 95 to 5 parts by weight, more preferably 30 to 70 parts by weight: 70 to 30 parts by weight. When the content is within the above range, the physical properties of the polymer composite particles produced and the feeling of use when applied to cosmetics are good, if content of the multifunctional crosslinking monomer is less than 5 parts by weight, solvent resistance, heat resistance and mechanical properties of the particles are degraded.

In the present invention, a commonly used method can be used for dissolution and dispersion, and the initiator to be added can be used without any particular limitation as long as the initiator can form a free radical. Specific examples include peroxide-based ones such as benzoyl peroxide, lauryl peroxide, cumene hydroperoxide, methyl ethyl ketone peroxide, and t-butyl hydroperoxide; azo compounds such as 2,2′-azobisisobutylonitrile, 2,2′-azobis-2,4-dimethylvaleronitrile and 2,2′-azobis-2-methylisobutyronitrile, or mixtures thereof. The initiator is preferably used in an amount of 0.1 to 10 parts by weight, preferably 0.5 to 5 parts by weight, based on 100 parts by weight of the radical polymerizable monomer and the multifunctional crosslinking monomer. Within the above range, an yield can be increased, heat generation can be suppressed during the polymerization processing, and a particle size can be easily controlled.

### b) Mixing Aqueous Phase in Emulsifier and Emulsifying to Form Droplets

An aqueous phase of b) may be prepared by dissolving an ionic imparting substance and a dispersion stabilizer in water. The water may be ion-depleted water or distilled water, and the ionic imparting substance is a substance that is dissolved in water to exhibit polarity, and as a specific example, ionic imparting substance may be selected from a group formed of NaNO₃, NaCl, NaOH, Na₂SO₄, NaNO₂, Na₂CO₃, Na₃PO₄, KNO₃, KCl, KOH, K₂SO₄, K₂CO₃, K₃PO₄, NH₄NO₃, NH₄Cl, NH₄OH, (NH₄)₂SO₄, (NH₄)₃PO₄, Mg(NO₃)₂, MgCl₂, Ca(NO₃)₂, CaCl₂, Ba(NO₃)₂, and BaCl₂. Preferably, the ionic-imparting substance is used in an amount of 10 to 30 parts by weight based on 100 parts by weight of the total of the radical polymerizable monomer and the multifunctional crosslinking monomer. More preferably, the ionic imparting substance is 20 to 30 parts by weight are used per 100 parts by weight of the total amount of the radical polymerizable monomer and the multifunctional crosslinking monomers. If the amount is within the above range, a matrix of the polymer to be polymerized afterwards can contain a large amount of the organic sunscreen agents desired in the present invention, and exposure of the organic sunscreen agent to the surface of the composite particles can be minimized.

The dispersion stabilizer is used to prevent aggregation and precipitation of particles formed in the polymerization reaction, and specific examples thereof include cellulose derivatives such as methyl cellulose, ethyl cellulose and hydroxypropyl cellulose; polyvinyl alcohol, polyvinyl methyl ether, polyacrylic acid, polyvinyl acetate or copolymers of polyvinyl pyrrolidone and vinyl acetate; colloidal silica, and the like. The dispersion stabilizer may be included in an amount of 0.1 to 20 parts by weight based on 100 parts by weight of the sum of the radical polymerizable monomer and the multifunctional crosslinking monomer. If concentration is less than 0.1 part by weight, there is a problem of flocculation. If the concentration is more than 20 parts by weight, a dispersion stabilizer may be gelated and a side reaction may occur.

The water may be used in an amount sufficient to dissolve the ionic imparting substance and the dispersion stabilizer.

The emulsion and droplets of b) may be formed by a commonly used method.

### c) Polymerizing Droplet to Prepare Polymer Composite Particles

The polymerization of c) may be performed by a polymerization method commonly used in the art. As a specific example, suspension polymerization may be applied. During the polymerization, the polymerization may proceed at 50° C. to 90° C. for 5 hours to 50 hours. This is because a high conversion rate can be obtained. At this time, if a stirring speed is too high in the polymerization reaction, it may be preferable to maintain an appropriate stirring speed because aggregation may occur or a particle size may be changed to cause a problem in size control.

### d) Washing and Drying Polymer Composite Particles Prepared

In d), the polymer composite particles prepared in d) are washed and dried.

Washing is performed so as to reduce the amount of unreacted monomers present in the particles after filtration of the synthesized particles and to remove impurities such as initiators remaining after the polymerization reaction to prevent discoloration due to impurities even if the temperature during drying increases, alcohols (such as methyl alcohol, ethyl alcohol, butyl alcohol, amyl alcohol, octyl alcohol, or benzyl alcohol), polyhydric alcohols (ethylene glycol, propylene glycol, glycerin, and the like), EDTA, aqueous solutions thereof, water, and the like can be used. In this case, the aqueous solution is preferably used in an amount of 1 to 30% by weight of the washing solution, and if used in the range, the removal efficiency of impurities can be increased.

Drying is preferably performed in a vacuum state at a temperature lower than a glass transition temperature in consideration of the glass transition temperature of the particles after removal of the impurities and during washing.

A method of preparing the polymer composite particles according to the present invention enables a large amount of solid organic sunscreen agent to be incorporated in a polymer matrix polymerized by using an ionic imparting substance in an amount of 40 to 50 wt %, and enables the organic sunscreen agent not to be exposed to the surface of the composite particles, and thereby, it is possible to provide the skin with stability while having significantly improved sunscreen function as compared with the composite particles of related art.

The present invention also provides a cosmetic composition including the polymer composite particle containing the organic sunscreen agent as an active ingredient.

A cosmetic composition can be used by adding the polymer composite particles to various formulations of the cosmetic composition, and the cosmetic composition preferably contains 0.01-30% by weight based on the total weight of the composition. In the present invention, formulation of the cosmetic material is not particularly limited.

The cosmetic composition according to the present invention has a very good feeling of use and the problem of skin troubles is remarkably reduced because the organic sunscreen agent is embedded in the polymer matrix. Particularly, compared with polymer composite particles of related art, cosmetic composition is excellent in light stability despite containing a large amount of organic sunscreen agent, and has excellent sunscreen properties for a UV-B region at the same time as a UV-A region.

Hereinafter, the present invention will be described in more detail with reference to the following examples. However, the scope of the present invention is not limited to the following examples.

### Embodiment 1

9 g of PVA and 75 g of NaCl were dissolved in 2000 g of deionized water to prepare a water phase. After 270 g of MMA (methyl methacrylate) and 30 g of EDGMA (ethylene glycol dimethacrylate) were added to the emulsion and stirred for 5 minutes, 163.5 g of avobenzone and 81.75 g of EHT (ethylhexyl triazine) were added 30 minutes, and 4 g of initiator 2,2′azobis-(2,4-dimethylvaleronitrile) was added thereto and mixed.

Thereafter, the emulsion is emulsified for 40 minutes by using an emulsifier. After the emulsion is heated, the emulsion is polymerized at 65° C. for 20 hours. The resultant polymer particles are dehydrated using a vacuum filter, is washed several times, and is vacuum-dried to obtain final polymer composite particles.

### Embodiment 2

The procedure of Embodiment 1 is repeated except that BHT (Bis-Ethylhexyloxyphenol Methoxyphenyl Triazine) is used instead of EHT in Embodiment 1.

### Embodiment 3

The procedure of Embodiment 1 is repeated except that EHT (54.5 g) and BEMT (27.25 g) are used instead of 81.75 g of EHT in Embodiment 1.

### Embodiment 4 to Embodiment 6

Embodiments 4 to 6 are performed in the same manner as in Embodiment 1, except that CaCl₂ (Embodiment 4), KCl (Embodiment 5) and MgCl₂ (Embodiment 6) are used instead of NaCl as the ionic imparting substance in Embodiment 1.

### Comparative Example 1

The procedure of Comparative Example 1 is repeated except that NaCl is not used in Embodiment 1.

### Comparative Example 2

Comparative Example 1 is performed in the same manner as in Embodiment 1, except that NaCl is not used and EHT is not used in Embodiment 1.

### Test Example 1: Photograph of Polymer Composite Particles

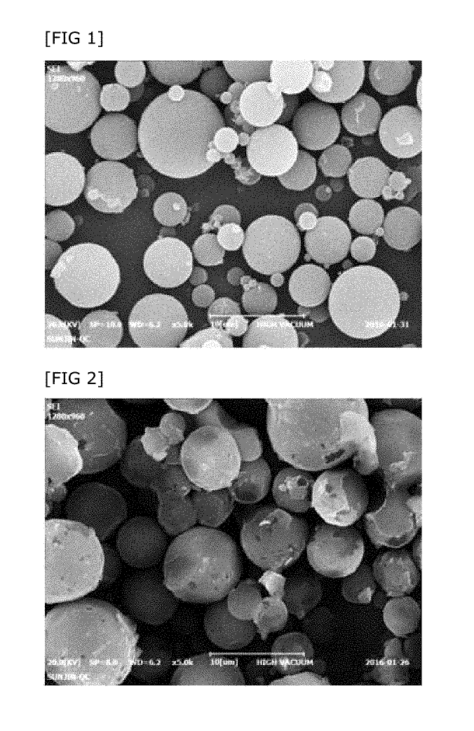

The polymer composite particles prepared in Embodiment 1 and Comparative Example 1 are taken at a magnification of 5000 times. FIG. 1 is a photograph of the polymer composite particle of Embodiment 1, and FIG. 2 is a photograph of the polymer composite particle of Comparative Example 1. As illustrated in FIG. 1 and FIG. 2, the polymer composite particle of Embodiment 1 according to the present invention has a clear and smooth surface of the composite particle in which the organic sunscreen agent is embedded inside the composite particle, but in the polymer composite particle of Comparative Example 1, a surface of the composite particle is in contrast to the composite particles of Embodiment 1, because the organic sunscreen agent is present in large amounts on the surface of the composite particle.

### Test Example 2: Iron Test

The polymer composite particle prepared in Embodiment 1 and Comparative Example 1 are tested for iron content. Specifically, 2 g of a sample is put into a 100 ml transparent sample bottle, 0.1 g of an iron standard solution (1000 ppm) is added, and 98 g of distilled water is added thereto, followed by shaking and even dispersion.

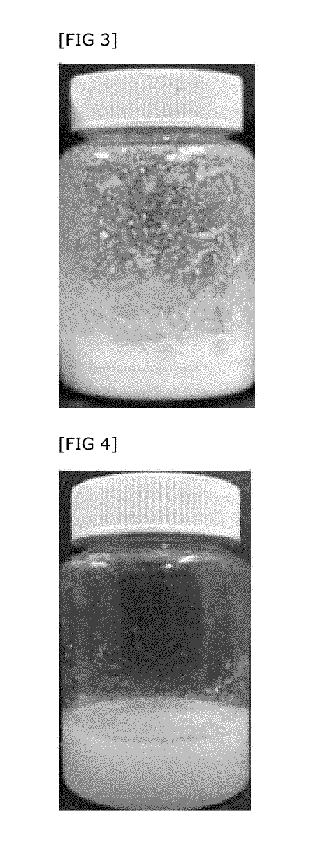

FIG. 3 is a photograph of a result of iron particle test of the polymer composite particle of Embodiment 1, and FIG. 4 is a photograph of an iron particle test result of the polymer composite particle of Comparative Example 1. FIG. 3 illustrates that the organic sunscreen agent does not exposed to the outside of the polymer composite particle while maintaining an original color, and FIG. 4 illustrates that the organic sunscreen agent are exposed the outside of the polymer composite particle.

### Characteristics and Sunscreen Function Experiment

### Formulation Examples 1 to 6 and Comparative Example 2

In order to confirm sunscreen function of the polymer composite particle prepared in Embodiments 1 to 6, sunscreen cosmetics in the form of W/O emulsion are prepared using the composite particles prepared in Embodiments 1 to 6 and Comparative Example 1, respectively, and a basic composition is illustrated in Table 1 below.

TABLE 1

| | Weight (parts by weight) | | | | | | | |
|---|---|---|---|---|---|---|---|---|
| | | | | | | | Comparative | Comparative |
| Composition | Example 1 | Example 2 | Example 3 | Example 4 | Example 5 | Example 6 | example 1 | example 2 |
| Alkylbenzoate | 42.0 | 42.0 | 42.0 | 42.0 | 42.0 | 42.0 | 42.0 | 42.0 |
| Silicone | 4.0 | 4.0 | 4.0 | 4.0 | 4.0 | 4.0 | 4.0 | 4.0 |
| emulsifier | | | | | | | | |
| (KF6027) | | | | | | | | |
| Bentonite | 2.0 | 2.0 | 2.0 | 2.0 | 2.0 | 2.0 | 2.0 | 2.0 |
| Polymer | 12.0 | 12.0 | 12.0 | 12.0 | 12.0 | 12.0 | — | — |
| composite | | | | | | | | |
| particles | | | | | | | | |
| obtained in | | | | | | | | |
| embodiment | | | | | | | | |
| Polymer | — | — | — | — | — | — | 12.0 | 12.0 |
| composite | | | | | | | | |
| particle obtained | | | | | | | | |
| in | | | | | | | | |
| comparative | | | | | | | | |
| example | | | | | | | | |
| Distilled | 20.0 | 20.0 | 20.0 | 20.0 | 20.0 | 20.0 | 20.0 | 20.0 |
| water | | | | | | | | |

The W/O emulsions prepared in Formulation Examples 1 to 6 are measured for their sunscreen function with an SPF 290 (Sun Protection Factor) analyzer from optometrix (USA). The results are illustrated in Table 2 below.

In addition, 12% by weight of powders of Embodiments 1 to 6 are added to prepare W/O type cream, and sunscreen function is measured. As a result, a very high PF (protection factor) value and a very high SPF (Sun Protection Factor) are obtained.

TABLE 2

| | Embodiment | | | | | | Comparative | Comparative |
|---|---|---|---|---|---|---|---|---|
| | 1 | Embodiment 2 | Embodiment 3 | Embodiment 4 | Embodiment 5 | Embodiment 6 | example 1 | example 2 |
| weight | 12 | 12 | 12 | 12 | 12 | 12 | 12 | 12 |
| ratio | | | | | | | | |
| of | | | | | | | | |
| polymer | | | | | | | | |
| composite | | | | | | | | |
| particles | | | | | | | | |
| containing | | | | | | | | |
| sunscreen | | | | | | | | |
| agent | | | | | | | | |
| (%) | | | | | | | | |
| SPF | 53.32 | 59.28 | 59.18 | 54.38 | 56.29 | 53.27 | 49.17 | 15.98 |
| UVA | 96.58 | 83.59 | 83.68 | 91.86 | 88.57 | 84.35 | 78.89 | 72.82 |
| PF | | | | | | | | |

### Test Example 2: Skin Irritation Test

Twenty test subjects were skin-patch-tested using the sunscreen cosmetics of Formulation Example 1 and Comparative Example 1 to test skin irritation. The results are illustrated in Table 3.

TABLE 3

| | Product of | Product of |
|---|---|---|
| | comparative | formulation |
| | example 1 | example 1 |
| +++ | | |
| ++ | 7 | |
| + | 11 | 2 |
| ± | 2 | 12 |
| − | | 6 |
| Total number | | |
| of people | | |

※determination standard
+++: strong trouble,
++: weak trouble,
+: slight trouble,
±: normal,
−negative

As can be seen from the above Table 3, it is confirmed that Formulation Example 1 is significantly superior to Comparative Example 1 in feeling.

### Formulation Example 7

The sunscreen cosmetics in the form of a W/O emulsion are prepared using the polymer composite particles prepared in Example 1, and the basic compositions are illustrated in Table 8 below.

TABLE 4

| | Weight(parts by weight) |
|---|---|
| composition | Formulation example7 W/O |
| Micro crystal line wax | 3.0 |
| Liquid paraffin | 7.0 |
| Silicon oil | 6.0 |
| Cyclopenta silicon | 6.0 |
| Paraben | 0.1 |
| Cetyl dimethicone copolyol | 2.0 |
| Polymer composite particles | 8.33 |
| obtained in Embodiment 1 | |
| Ethyl hexyl methoxyninite | 7.0 |
| Butyleneglycol | 5.0 |
| water | 61.4 |

### Formulation Example 8

The sunscreen cosmetics in the form of a W/O emulsion are prepared using the polymer composite particles prepared in Embodiment 1, and the basic compositions are illustrated in Table 5 below.

TABLE 5

| | Weight (parts by weight) |
|---|---|
| composition | Formulation example8 W/O |
| Beads wax | 2.0 |
| Stearyl alcohol | 1.5 |
| Stearic acid | 0.5 |
| Squalane | 10.0 |
| Polyoxyethylene ethyl ether | 1.0 |
| Propylene glycol monostearate | 3.0 |
| Ethyl hexylmethoxycinnamate | 7.0 |
| Paraben | 0.1 |
| Polymer composite particles | 8.33 |
| obtained in embodiment 1 | |
| Propylene glycol | 8.0 |
| glycerin | 4.0 |
| Preethylamine | 1.0 |
| water | 59.4 |

It will be understood by those skilled in the art that various changes in form and details may be made therein without departing from the spirit and scope of the invention as defined by the appended claims.

What is claimed is:

1. A polymer composite particle comprising:
a spherical particle having a solid organic sunscreen agent which is embedded between polymer matrixes by 40 to 50 wt %.

2. The polymer composite particles according to claim 1, wherein a size of the composite particle is 1 to 100 μm.

3. The polymer composite particles according to claim 1, wherein the organic sunscreen agent includes at least two kinds of sunscreen agents.

4. The polymer composite particles according to claim 3, wherein the organic sunscreen agent includes 50 to 90 parts by weight of avobenzone and 10 to 50 parts by weight of a triazine-based sunscreen agent

5. The polymer composite particles according to claim 4, wherein the triazine-based sunscreen agent is selected from groups which are formed of BEMT(Bis-Ethylhexyloxyphenol Methoxyphenyl Triazine), Ethylhexyl Triazone, and diethylhexyl butamido triazone.

6. A method of preparing polymer composite particles, the method comprising:
a) dissolving and dispersing an organic sunscreen agent in a radical polymerizable monomer and a multifunctional crosslinking monomer in an emulsifying agent and adding an initiator to prepare an oil phase;
b) mixing and emulsifying an aqueous phase containing an ionic imparting substance, a dispersion stabilizer, and water in the emulsifying agent to form droplets;
c) polymerizing the droplets to prepare polymer composite particles; and
d) washing and drying the polymer composite particles which are prepared.

7. The method of preparing a polymer composite particle according to claim 6, wherein the ionic-imparting substance is 10 to 30 parts by weight per 100 parts by weight of a sum of the radical polymerizable monomer and the multifunctional crosslinking monomer.

8. The method of preparing a polymer composite particle according to claim 6, wherein one or more of the ionic imparting substance are selected from a group which is formed of NaNO₃, NaCl, NaOH, Na₂SO₄, NaNO₂, Na₂CO₃, Na₃PO₄, KNO₃, KCl, KOH, K₂SO₄, K₂CO₃, K₃PO₄, NH₄NO₃, NH₄Cl, NH₄OH, (NH₄)₂SO₄, (NH₄)₃PO₄, Mg(NO₃)₂, MgCl₂, Ca(NO₃)₂, CaCl₂, Ba(NO₃)₂, and BaCl₂.

9. A cosmetic composition comprising:
the polymer composite particles according to claim 1 as an active ingredient.
